(19) United States
(12) Patent Application Publication
(10) Pub. No.: US 2004/0198633 A1
(43) Pub. Date: Oct. 7, 2004

(54) Identifying parasitic fungi of the organism, and treatment thereof

(76) Inventor: Roumen Antonov, Paris (FR)

Correspondence Address: YOUNG & THOMPSON, 745 SOUTH 23RD STREET 2ND FLOOR, ARLINGTON, VA, 22202

(21) Appl. No.: 10/478,508

(22) PCT Filed: May 23, 2002
(86) PCT No.: PCT/FR02/01739

(30) Foreign Application Priority Data
May 23, 2001 (FR) 01/06775
Jul. 11, 2001 (FR) 01/09203

(51) Int. Cl. A61K 31/00

(52) U.S. Cl. 514/1

(57) ABSTRACT

The invention concerns the use of antifungal agents, in particular those active on fungi of genus Candida, for preparing medicines for preventing or treating pathologies related to the presence in the human organism the following fungi: Entomocorticum sp., Stereum annosum, Phellinus igniarus, Peniophora pithya, Peniophora pieceae, Xilaria longipes, Rosellinia arcuata, Rosellinia necatrix, Multiclavula vernalis, Hericium coralloides, Hericium abietis, Hericium erinaceum, Hericium rivularis, Tricholoma terreum.

A subject of the present invention is revealing parasitic fungi in the human organism, and the use of antifungals, such as those active on fungi of the genus Candida, for the preparation of medicaments intended for the prevention or treatment of pathologies, in humans or animals, linked to the presence in the human or animal organism of these fungi.

Recent experiments undertaken on samples of human blood vessels, comprising if appropriate atheromatous plaques, have allowed the Inventor to unambiguously conclude the presence of at least one fungus chosen from Entomocorticum sp., Stereum annosum, Phellinus igniarus, Peniophora pithya, Peniophora piceae, Xilaria longipes, Rosellinia arcuata, Rosellinia necatrix, Multiclavula vernalis, Hericium coralloides, Hericium abietis, Hericium erinaceum, Hericium rivularis, Tricholoma terreum, in the human organism, in particular in the atheromatous plaques, and other sclerotic tissues, namely of fungi of neighbouring species having a common biotope, and more particularly a common biological activity, namely the degradation of wood.

A subject of the invention is the use of antifungals, such as those which are active on fungi of the genus Candida, for the preparation of a medicament intended to eliminate from the human or animal organism at least one fungus, close or identical to the species involved in the degradation of wood, said fungus being chosen from the following: Entomocorticum sp., Stereum annosum, Phellinus igniarus, Peniophora pithya, Peniophora piceae, Xilaria longipes, Rosellinia arcuata, Rosellinia necatrix, Multiclavula vernalis, Hericium coralloides, Hericium abietis, Hericium erinaceum, Hericium rivularis, Tricholoma terreum, or intended for the prevention or treatment of pathologies linked to the presence in the human or animal organism of at least one fungus defined above.

A more particular subject of the invention is the use of the abovementioned antifungals, for the preparation of a medicament intended to eliminate from the human or animal organism, or intended for the prevention or treatment of pathologies linked to the presence in the human or animal organism of fungi chosen from Stereum annosum and/or Phellinus igniarus, if appropriate in association with at least one fungus or all of the fungi mentioned above.

The invention relates more particularly to the use of the abovementioned antifungals, for the preparation of a medicament intended to eliminate from the human or animal organism, or intended for the prevention or treatment of pathologies linked to the presence in the human or animal organism, of the filamentous fungus Phellinus igniarus deposited on Jul. 3, 2001 at the “Collection Nationale de Cultures de Microorganismes” (CNCM) of Pasteur Institute (“Institut Pasteur”) under number I-2690, and/or the filamentous fungi Stereum annosum deposited on Jul. 3, 2001 at the CNCM under number I-2691, and/or filamentous fungi Entomocorticum sp. deposited on Jul. 3, 2001 at the CNCM under number I-2692.

A more particular subject of the invention is the use of antifungals as defined above and hereafter, for the preparation of a medicament intended for the prevention or treatment of tissue scleroses or necroses, or pathologies originating from these scleroses or necroses, and being linked to the presence in the human or animal organism of at least one fungus such as described above.

A more particular subject of the invention is the above mentioned use of antifungals as described above, for the preparation of a medicament intended for the prevention or treatment of pathologies linked to the development of scleroses or necroses in the organism, namely of a medicament intended to prevent or to resorb the scleroses or necroses resulting from the deposit and accumulation of cellular debris in the tissues, as well as their retention in the form of clusters by the fungi as described above, developing in these sclerotic or necrotic structures.

The invention also relates to the use of antifungals as defined above and hereafter, for the preparation of a medicament intended for the prevention or treatment of scleroses or necroses in the blood vessels linked to the presence in these blood vessels of the human or animal organism, of at least one fungus such as described above, and more particularly of atherosclerosis as well as different pathologies linked to the abovementioned atherosclerosis, such as myocardial infarction, phlebitis, and cerebral vascular thrombosis.

A subject of the invention is also the use of antifungals as defined above and hereafter, for the preparation of a medicament intended for the prevention or treatment of scleroses or necroses of the skin, such as eczema, psoriasis, erythemas, ichthyosis, linked to the presence in the human or animal organism, of at least one fungus such as described above, or also of a medicament intended for the cleaning of necrotic scars and wounds and the elimination of gangrene in particular in the case of ulcers (for example of the legs) of venous origin, eschars, trauma, chronic, ulcerated or atonic wounds, superficial and deep burns, cutaneous necroses caused by trauma, in which the abovementioned fungus or fungi are found.

The invention also relates to the use of antifungals as defined above and hereafter, for the preparation of a medicament intended for the prevention or treatment of scleroses or necroses of the joints, in particular in the case of arthrosis, linked to the presence in the human or animal organism of at least one fungus such as described above.

A subject of the invention is also the use of antifungals as defined above and hereafter, for the preparation of a medicament intended for the prevention or treatment of pulmonary scleroses or necroses, and more particularly of pulmonary pathologies of sclerotic origin such as asthma or spasmodic cough, linked to the presence in the human or animal organism of at least one fungus such as described above.

By antifungals active on the fungi defined above, is meant more particularly those active on the fungi of the genus Candida, in particular any compound active on Candida Albicans, or on any derived species or sub-species, in particular by mutation, of the abovementioned fungi.

Amongst the antifungals capable of being used within the scope of the present invention, the following can be mentioned:

Flucytosine (DCI),

antifungal contact antibiotics of the polyene family,

antifungals of the imidazoles group, such as:

Miconazole (DCI),

Ketoconazole (DCI),

Fluconazole (DCI) (bistriazolated antifungal).

A more particular subject of the invention is the above-mentioned use of antifungal contact antibiotics of the polyene family, and more particularly those extracted from bacteria cultures of the genus Streptomyces, such as:

Amphotericin B (DCI) extracted from culture of Streptomyces nodosus,

Nystatin (DCI) extracted from culture of Streptomyces noursei,

or any derivative of these antifungals, such as those capable of being obtained by chemical synthesis.

By way of illustration, the polyenes able to be used within the scope of the present invention are the following:

tetraenes of formulae 1 which follow:

nystatin of formula 1a

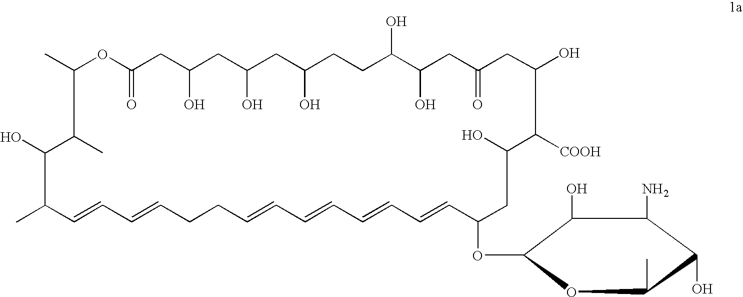

pimarycin of formula 1b in which R═CH₃ and arenomycin in which R═CH₂—CH₂—CH₂—CH₃

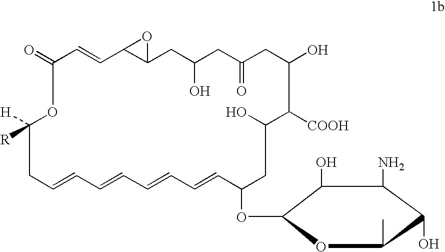

rimocide of formula 1c

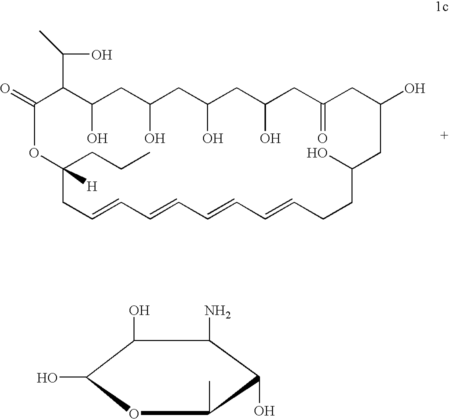

tetrin A of formula 1d in which R═H, and tetrin B in which R═OH

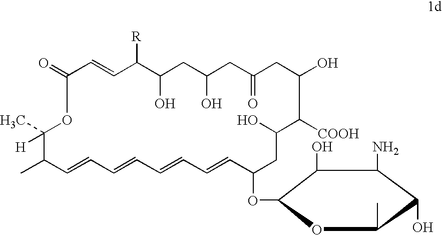

pentaenes of formulae 2 which follow:

philipine of formula 2a in which R═H, and pentamycin in which R═OH

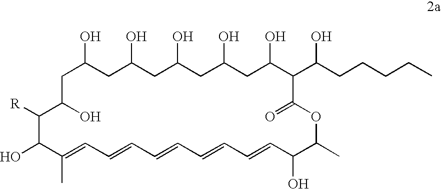

mycotitzine A of formula 2b in which R═H, and mycotitzine B of formula 2b in which R═CH₃

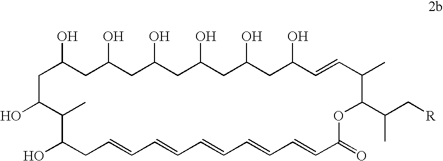

eirotitzine A of formula 2c in which R═CH₃, and eirotitzine B of formula 2c in which R═H

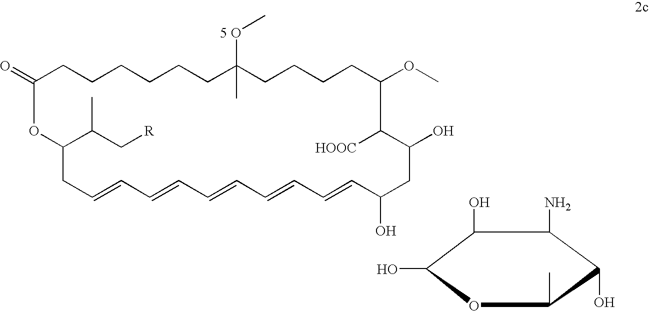

the compound of formula 2d which follows

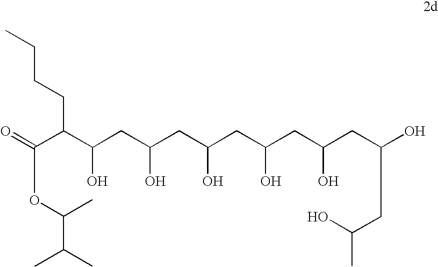

hexaenes of formulae 3 which follow:

dermostatin A of formula 3 in which R═CH₃, and dermostatin B of formula 3 in which R═CH₂—CH₃

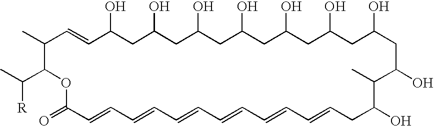

heptaenes of formulae 4 which follow:

amphotericin B of formula 4a

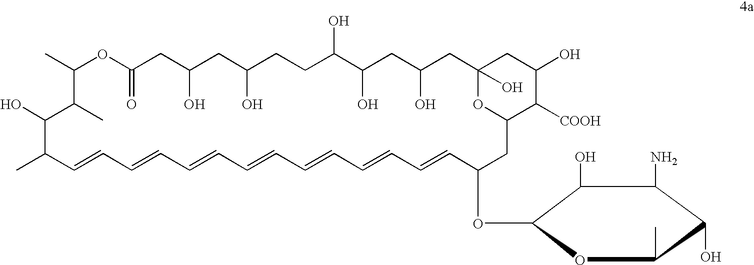

candidin of formula 4b

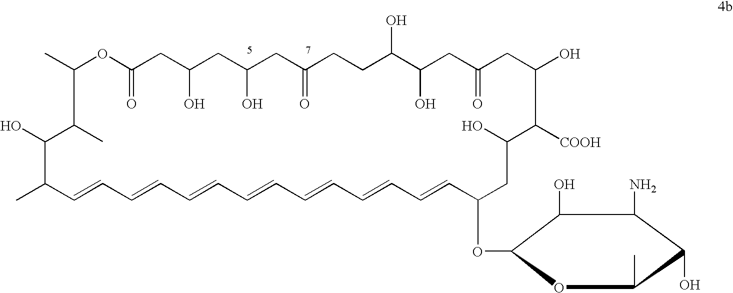

Advantageously, the antifungal used within the scope of present invention is Nystatin.

Advantageously also, the doses of antifungals used within the scope of present invention are approximately 2 to approximately 100 times, in particular approximately 5 to approximately 50 times, lower than the usual doses of these same antifungals (namely at usual doses of approximately 10 tablets of 500,000 IU per day for 10 days) in the treatment of candidiasis, in particular buccopharyngeal, intestinal or vaginal candidiasis.

Moreover, the treatment period is much longer, in particular at least approximately 5 times longer, in the case of the present invention, than in the case of the treatment of the abovementioned candidiasis, and is in particular more than at least approximately 2 months, in particular at least approximately 6 months.

By way of illustration, the doses used in the case of the use of antifungals, and more particularly of Nystatin, within the scope of present invention, are the following:

less than approximately 100,000 units per day in adults, in particular in persons of between approximately 20 years and approximately 30 to 40 years of age, or

between approximately 25,000 and approximately 50,000 units per day in persons of less than approximately 20 years, and in children,

between approximately 25,000 and approximately 125,000 units per week, advantageously approximately 25,000 units per week, in persons of more than 40 years of age, for an unlimited treatment period.

The doses indicated above, are administered once or more times daily for the duration of the treatment, or, preferably, administered once or more times daily spread out at intervals of approximately 3 to approximately 7 days for the duration of the treatment.

At the doses indicated above, the duration of the treatment is from approximately at least 6 months for children and persons of less than approximately 20 years of age, up to one or more years in persons of more than 20 years of age.

It can be noted that after such treatment periods, the immune system becomes capable of taking over against the development of these scleroses or necroses due to the development of fungi as described above, through an immunization process, for a period of between approximately 3 years and approximately 5 years. The treatment with the abovementioned antifungals can then cease.

In the case where this immunization were to disappear, the treatment using the abovementioned antifungals can then be resumed, preferably with doses approximately two times lower than those indicated above, and for a duration approximately two times shorter.

The doses and treatment periods indicated above allow the significant reduction of the quantities of fungi such as described above in the organism.

A subject of the invention is also, the above-mentioned use of antifungals as described above, at doses allowing the growth of said fungi in the organism to be stopped. In this case, the dose used in the use of the antifungals, and more particularly of Nystatin, is advantageously approximately 25,000 units per week (in one or more doses), in persons of more than 40 years of age, for an unlimited treatment period.

A subject of the invention is also the methods of treatment of the abovementioned pathologies, by administration to the patients capable of being treated, of the abovementioned antifungals, in particular at the doses indicated above, and for the abovementioned periods.

Advantageously, the abovementioned antifungals are used, in the present invention, for the preparation of medicaments capable of being administered by oral route, in particular in the form of tablets or of drinkable suspensions, or by injection.

A more particular subject of the invention is a method of in vitro diagnosis of pathologies linked to the presence in the human or animal organism of at least one fungus defined above, and more particularly of the abovementioned tissue scleroses or necroses, characterized in that it comprises the detection in an appropriate biological sample taken from a human or an animal, such as tissue or blood biopsies, of at least one fungus defined above, if appropriate after culturing said biological sample until the development and possible characterization of the fungus, or using molecular techniques employing nucleotide probes and/or nucleotide primers in PCR, or of appropriate antibodies, said probes, primers or antibodies being specific to the abovementioned fungi.

The invention relates yet more particularly to the use of antifungals as described above for the preparation of medicaments intended for the prevention or treatment of pathologies linked to the presence in the human or animal organism of at least one fungus defined above, and more particularly of the tissue scleroses or necroses mentioned above, and having been the object of a diagnosis according to the abovementioned method.

The invention is illustrated using Table I below which represents the numerical percentages of all the species isolated from biopsies of blood vessels of patients presenting, if necessary, atheromatous plaques, said species having been identified by sequencing.

TABLE 1

| | Numerical percentage of |
|---|---|
| SPECIES | all the species isolated |
| Entomocorticium sp. | 28.5% |
| Stereum annosum | 28.5% |
| Phellinus igniarus | 14% |
| Peniophora pithya, or P. piceae, | 7% |
| Xilaria longipes, or | 7% |
| Rosellinia arcuata, or R necatrix, | |
| Multiclavula vernalis, or | 14% |
| Hericium coralloides, or H. abietis, or | |
| H. erinaceum, or H. rivularis, or Tricholoma | |
| terreum, | |

1. Use of antifungals, such as those active on the fungi of the genus Candida, for the preparation of a medicament intended to eliminate from the human or animal organism at least one fungus chosen from the following: Entomocorticum sp., Stereum annosum, Phellinus igniarus, Peniophora pithya, Peniophora piceae, Xilaria longipes, Rosellinia arcuata, Rosellinia necatrix, Multiclavula vernalis, Hericium coralloides, Hericium abietis, Hericium erinaceum, Hericium rivularis, Tricholoma terreum, or intended for the prevention or treatment of pathologies linked to the presence in the human or animal organism of at least one fungus defined above.

2. Use of antifungals according to claim 1, for the preparation of a medicament intended to eliminate from the human or animal organism, or intended for the prevention or treatment of pathologies linked to the presence in the human or animal organism of fungi chosen from Stereum annosum and/or Phellinus igniarus, if necessary in association with at least one fungus.

3. Use of antifungals according to claim 1, for the preparation of a medicament intended to eliminate from the human or animal organism, or intended for the prevention or treatment of pathologies linked to the presence in the human or animal organism, of the filamentous fungi Phellinus igniarus deposited at the “Collection Nationale de Cultures de Microorganismes” (CNCM) of Pasteur Institute (“Institut Pasteur”) under the number I-2690, and/or of the filamentous fungi Stereum annosum deposited at the CNCM under number I-2691, and/or of filamentous fungi Entomocorticum sp. deposited at the CNCM under number I-2692.

4. Use of antifungals according to claim 1, for the preparation of a medicament intended for the prevention or treatment of tissue scleroses or necroses, or of pathologies originating from these scleroses or necroses, and being linked to the presence in the human or animal organism of at least one fungus.

5. Use of antifungals according to claim 1, for the preparation of a medicament intended for the prevention or the treatment:
of atherosclerosis linked to the presence in the blood vessels of the human or animal organism, of at least one fungus, as well as of different pathologies linked to the abovementioned atherosclerosis, such as myocardial infarction, phlebitis, and cerebral vascular thrombosis,
of scleroses or necroses of the skin, such as eczema, psoriasis, erythemas, ichthyosis, linked to the presence in the human or animal organism of at least one fungus or also of a medicament intended for the cleaning of necrotic scars and wounds and the elimination of gangrene in particular in the case of ulcers (for example of the legs) of venous origin, eschars, trauma, chronic, ulcerated or atonic wounds, superficial and deep burns, cutaneous necroses caused by trauma, in which the fungi are found,
of scleroses or necroses of the joints, in particular in the case of arthrosis, linked to the presence in the human or animal organism of at least one fungus,
of pulmonary pathologies of sclerotic origin, and more particularly of asthma or of spasmodic cough, linked to the presence in the human or animal organism of at least one fungus.

6. Use according to claim 1, of antifungals chosen from the following:
Flucytosine (DCI),
antifungal contact antibiotics of the polyene family,
antifungals of the imidazole group, such as:
Miconazole (DCI),
Ketoconazole (DCI),
Fluconazole (DCI) (bistriazolated antifungal).

7. Use according to claim 1, of antifungal contact antibiotics of the polyene family, and more particularly those extracted from bacteria cultures of the genus Streptomyces, such as:
Amphotericin B (DCI) extracted from culture of Streptomyces nodosus,
Nystatin (DCI) extracted from culture of Streptomyces noursei.

8. Use according to claim 1, of Nystatin.

9. Use according to claim 1, of antifungals, and more particularly of Nystatin, at the following doses:
less than approximately 100,000 units per day in adults, in particular in persons of between approximately 20 years and approximately 30 to 40 years of age, or
between approximately 25,000 and approximately 50,000 units per day in persons of less than approximately 20 years of age, and in children, or
between approximately 25,000 and approximately 125,000 units per week, advantageously approximately 25,000 units per week, in persons of more than 40 years of age, for an unlimited treatment period.

10. Use according to claim 9, at the rate of one or more daily doses for the duration of the treatment, or, preferably, at the rate of one or more doses per day spaced out at intervals of approximately 3 to approximately 7 days for the duration of the treatment, the duration of the treatment being from approximately at least 6 months for children and persons of less than approximately 20 years of age, up to one or more years in persons of more than 20 years of age.

11. Method of in vitro diagnosis of pathologies linked to the presence in the human or animal organism of at least one fungus defined in claim 1, characterized in that it comprises the detection in an appropriate biological sample, such as tissue or blood biopsies, of at least one abovementioned fungus.

12. Method of in vitro diagnosis according to claim 11, of pathologies originating from scleroses or necroses, and being linked to the presence in the human or animal organism of at least on fungus.

13. Use of antifungals such as defined in claim 1, for the preparation of medicaments intended for the prevention or treatment of pathologies linked to the presence in the human or animal organism of at least one fungus, such as tissue scleroses or necroses, and having been the object of a diagnosis.
